(19) United States
(12) Patent Application Publication
(10) Pub. No.: US 2021/0077642 A1
(43) Pub. Date: Mar. 18, 2021

(54) STERILIZER

(71) Applicants: Yoshihiko YAMAMOTO, Osaka (JP); Mayumi YAMAMOTO, Osaka (JP)

(72) Inventors: Yoshihiko YAMAMOTO, Osaka (JP); Mayumi YAMAMOTO, Osaka (JP)

(21) Appl. No.: 17/018,112

(22) Filed: Sep. 11, 2020

(30) Foreign Application Priority Data
Sep. 13, 2019 (JP) 2019-167735

(51) Int. Cl. A61L 2/10; A61L 2/26

(52) U.S. Cl. CPC A61L 2/10; A61L 2202/24; A61L 2202/11; A61L 2/26

(57) ABSTRACT

A sterilizer includes a casing having an inner wall that is treated with a material including titanium oxide and an LED element for emission of ultraviolet rays that is attached to the inner wall of the casing. Ultraviolet rays emitted from the LED element hit the inner wall of the casing to be reflected. An object to be sterilized that is placed in the casing is sterilized by the ultraviolet rays emitted from the LED element and the ultraviolet rays reflected from the inner wall.

### BACKGROUND

### Technical Field

The present invention relates to a sterilizer for sterilizing various objects to be sterilized.

### Description of Related Art

In hospitals, sterilizers are used for sterilizing medical equipment, clothing, etc. In research institutions and universities other than the hospitals, sterilizers are used to sterilize laboratory instruments, etc. In these facilities, sterilizers that perform sterilization by generating a high temperature and a high pressure such as an autoclave (a high-pressure steam sterilizer), a flared sterilizer or a dry sterilizer are mainly used. JP 2013-75046 A discloses a high-pressure steam sterilizer for sterilizing medical equipment, etc. using a high-pressure steam.

### SUMMARY

The size of the above-mentioned sterilizers such as an autoclave is large in order to generate a high temperature and a high pressure. Further, the power consumption of these devices are high. For example, in a case where the sterilizers are used in outdoor hospitals in areas where infectious diseases are prevalent, user friendliness of the sterilizers is improved if the devices are small and light. Further, it is difficult to ensure power supply in outdoor hospitals, so that the sterilizers desirably operate with low power consumption.

An object of the present invention is to provide a sterilizer that is small and requires low power consumption.

(1) A sterilizer according to one aspect of the present invention includes a casing having an inner wall that is treated with a material including titanium oxide, and an LED element for emission of ultraviolet rays that is attached to the inner wall of the casing. Thus, an object to be sterilized in the casing can be sterilized by the ultraviolet rays emitted from the LED element and the ultraviolet rays reflected from the inner wall of the casing.

(2) A metal thin film may further be formed on part of the inner wall. An oxidation-reduction reaction caused by titanium oxide is promoted by the metal thin film. The sterilization effects are improved by the oxidation-reduction reaction caused by titanium oxide. Further, the reflection efficiency of the ultraviolet rays is improved by the metal thin film.

(3) The metal may include gold. The oxidation-reduction reaction caused by titanium oxide is promoted by a gold thin film. The sterilization effects are improved by the oxidation-reduction reaction caused by titanium oxide. Further, reflection efficiency of the ultraviolet rays is improved by the gold thin film.

(4) The sterilizer may further include an LED supporter to which the LED element is attached, and an angle formed by an attachment surface of the LED supporter to which the LED element is attached and the inner wall may be smaller than 90°. Because the LED element attached to the attachment surface emits ultraviolet rays toward the inner wall, the ultraviolet rays can be efficiently reflected from the inner wall.

(5) The LED supporter may include a plurality of plates, and a cross section of the LED supporter may be formed to be a polygon by the plurality of plates. The entire circumference can be irradiated with ultraviolet rays by the LED element attached to each plate.

The present invention enables provision of a sterilizer that is small and requires low power consumption.

Other features, elements, characteristics, and advantages of the present invention will become more apparent from the following description of preferred embodiments of the present invention with reference to the attached drawings.

### BRIEF DESCRIPTION OF THE DRAWING

FIG. 1 is a diagram showing the appearance of a sterilizer according to an embodiment;

FIG. 2 is an exploded view of the sterilizer;

FIG. 3 is a perspective view of a germicidal lamp;

FIG. 4 is a cross sectional view of the germicidal lamp;

FIG. 5 is a diagram showing the emission range of an LED;

FIG. 6 is a diagram showing an inner wall of an inner casing of the sterilizer;

FIG. 7 is a diagram showing the emission directions of ultraviolet rays emitted by the germicidal lamp; and

FIG. 8 is a diagram showing netted racks attached to the inner casing of the sterilizer.

### DETAILED DESCRIPTION

### (1) Overall Configuration of Sterilizer

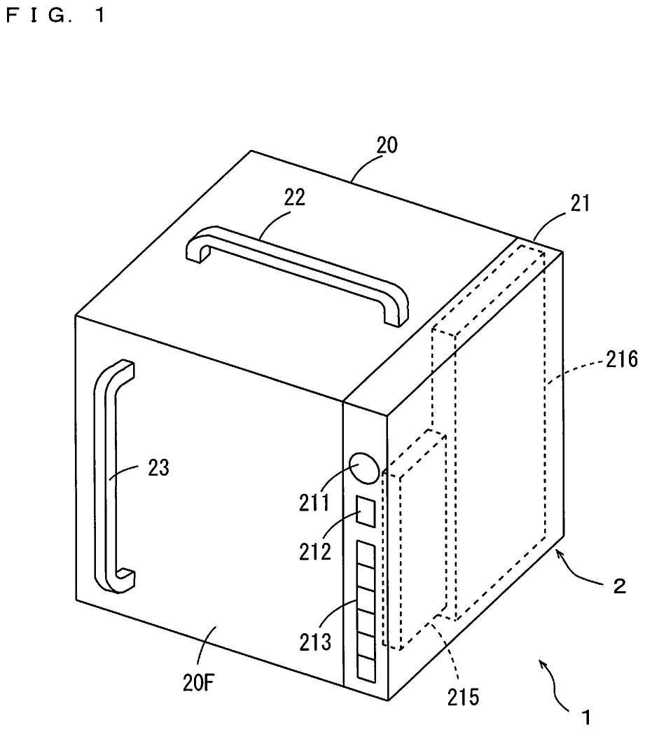

The configuration of a sterilizer 1 will be described below in detail in the present embodiment with reference to the drawings. FIG. 1 is a diagram showing the appearance of the sterilizer 1. As shown in FIG. 1, the sterilizer 1 includes an outer casing 2 having a substantially rectangular solid shape. The size of the sterilizer 1 of the embodiment is 30 cm in length, 35 cm in width and 30 cm in depth. However, the size of the sterilizer 1 is not limited in particular and suitably designed in accordance with the size of an object to be sterilized and the use of the sterilizer 1 such as necessity to carry.

The outer casing 2 of the sterilizer 1 includes a sterilization chamber storage 20 and a controller storage 21. A holder 22 is provided on an upper surface of the sterilization chamber storage 20. A user can carry the sterilizer 1 while holding the holder 22. A holder 23 is provided on a front surface of the sterilization chamber storage 20. The user can put an object to be sterilized into and out from the sterilization chamber 5 by opening a door 20F of the sterilization chamber storage 20 while holding the holder 23.

An ON-OFF button 211, a time display 212 and a lighting state display 213 are provided at the controller storage 21. Further, a controller 215 and a battery 216 are stored in the controller storage 21.

The ON-OFF button 211 is a button for instructing the controller 215 to start or stop a sterilization process. When the door 20F of the sterilization chamber storage 20 is open, even in a case where the ON-OFF button 211 is depressed, the controller 215 does not start the sterilization process. The ON-OFF button 211 can also be operated in a rotational manner. When the ON-OFF button 211 is operated in a rotational manner, an instruction in regard to a sterilization period of time can be provided to the controller 215. In the time display 212, the sterilization period of time designated using the ON-OFF button 211 is displayed. When the sterilization process is started by depression of the ON-OFF button 211, a remaining sterilization period of time is displayed in the time display 212 while being updated. The lighting states of six germicidal lamps, described below, are displayed in the lighting state display 213. The user can confirm whether the six germicidal lamps are operating normally by referring to the lighting state display 213.

### (2) Configurations of Outer Casing and Inner Casing

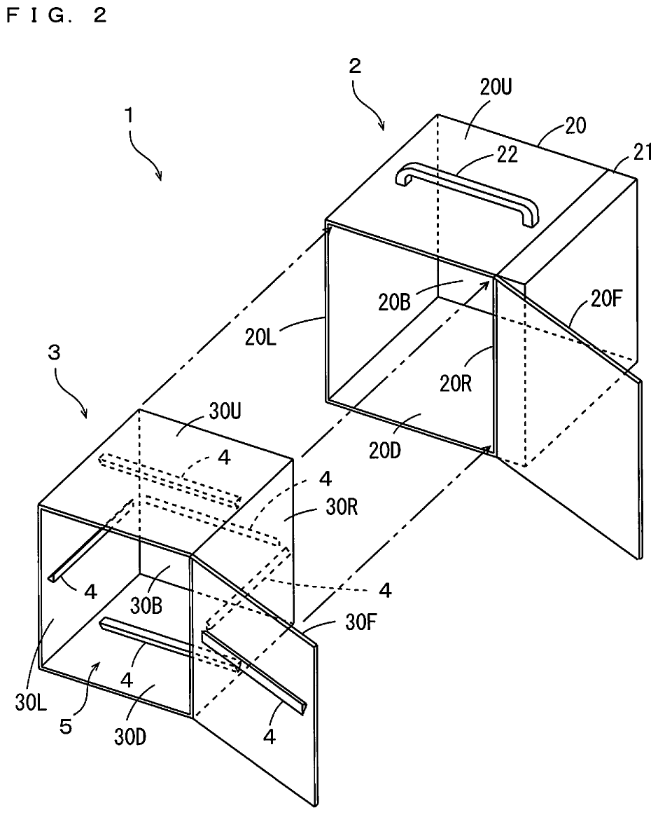

FIG. 2 is an exploded view of the sterilizer 1. The sterilizer 1 includes the outer casing 2 and an inner casing 3 as shown in FIG. 2. The inner casing 3 is stored in the outer casing 2. First, the configuration of the outer casing 2 will be described.

As described above, the outer casing 2 includes the sterilization chamber storage 20 and the controller storage 21. The sterilization chamber storage 20 has a substantially rectangular solid shape and has the hollow structure. The sterilization chamber storage 20 includes an upper wall 20U, a lower wall 20D, a right wall 20R, a left wall 20L, the door (front wall) 20F and a rear wall 20B. In the present embodiment, the upper wall 20U, the lower wall 20D, the right wall 20R, the left wall 20L, the door 20F and the rear wall 20B are members made of aluminum. The upper wall 20U, the lower wall 20D, the right wall 20R, the left wall 20L, the door 20F and the rear wall 20B constitute the hollow structure having a substantially rectangular solid shape, and the inner casing 3 is storable inside.

The door 20F is rotatably attached to the front end of the right wall 20R. The holder 22 is attached to the upper surface of the upper wall 20U.

The controller 215 and the battery 216 are stored in the controller storage 21. The controller 215 includes a CPU and the like and controls the sterilizer 1 as a whole. The controller 215 receives an operating instruction from the ON-OFF button 211. The controller 215 notifies the time display 212 and the lighting state display 213 of an operation state of the sterilizer 1. A lithium-ion battery is used as the battery 216 in the present embodiment. The battery 216 supplies electric power to the controller 215, the germicidal lamps, etc.

Next, the configuration of the inner casing 3 will be described. The inner casing 3 has a substantially rectangular solid shape and has the hollow structure. The inner casing 3 includes an upper wall 30U, a lower wall 30D, a right wall 30R, a left wall 30L, a door (front wall) 30F and a rear wall 30B. In the present embodiment, the upper wall 30U, the lower wall 30D, the right wall 30R, the left wall 30L, the door 30F and the rear wall 30B are members made of aluminum. Further, an inner wall of each wall is treated with a material including titanium oxide as described below. The upper wall 30U, the lower wall 30D, the right wall 30R, the left wall 30L, the door 30F and the rear wall 30B constitute the hollow structure having a substantially rectangular solid shape, and the hollow is the sterilization chamber 5.

The inner casing 3 that is constituted as described above is stored in the outer casing 2 as shown in FIG. 2, whereby the sterilizer 1 is constituted. The inner casing 3 is stored in the outer casing 2, and then the inner casing 3 and the outer casing 2 are fixed to each other by fixtures (not shown). Further, the door 20F and the door 30F may be fixed to each other. Thus, the door 20F and the door 30F can be integrally opened and closed.

### (3) Configuration of Germicidal Lamps

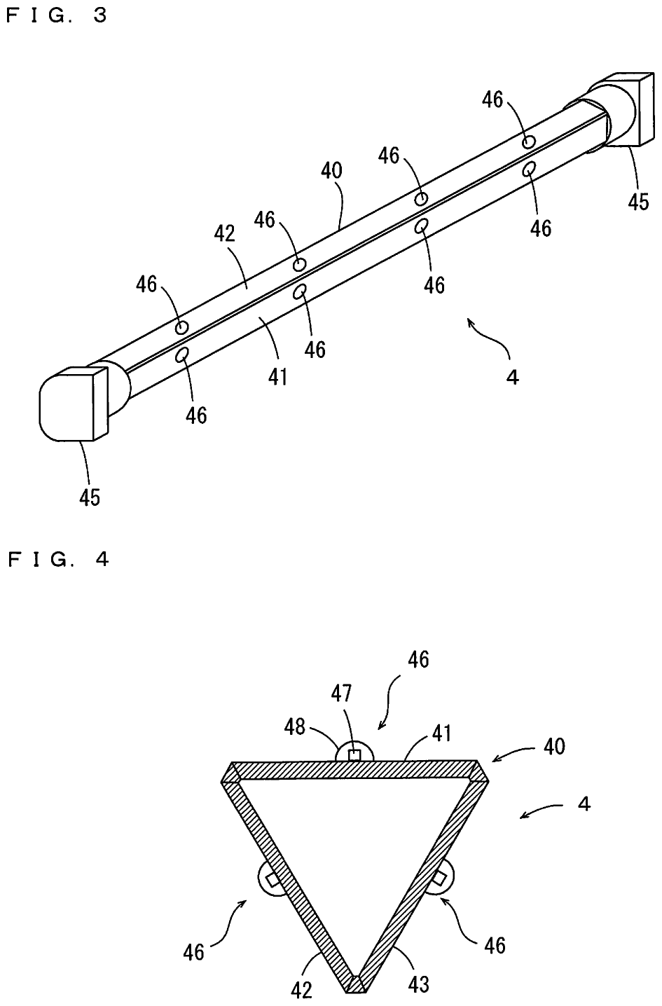

FIG. 3 is a perspective view of a germicidal lamp 4 according to the embodiment. As shown in FIG. 3, the germicidal lamp 4 according to the embodiment has an elongated shape similarly to a fluorescent lamp. In the present embodiment, the length of the germicidal lamp 4 is substantially 25 cm. As shown in FIG. 2, the six germicidal lamps 4 are stored in the sterilization chamber 5 in the inner casing 3. That is, one sterilization lamp 4 is attached to each of the upper wall 30U, the lower wall 30D, the right wall 30R, the left wall 30L, the door (front wall) 30F and the rear wall 30B.

As shown in FIG. 3, the germicidal lamp 4 includes an elongated LED supporter 40 and connection portions 45 that are provided at both ends of the LED supporter 40. FIG. 4 is a cross sectional view of the LED supporter 40 of the germicidal lamp 4. That is, FIG. 4 is a cross sectional view of the LED supporter 40 that is cut perpendicularly to the longitudinal direction of the LED supporter 40. The LED supporter 40 is constituted by three elongated plates 41, 42, 43. As shown in FIG. 4, the three plates 41, 42, 43 are assembled such that the cross section is a substantially equilateral triangle.

As shown in FIG. 3, four light emitters 46 are arranged at each of the plates 41, 42. While not shown in FIG. 3, four light emitters 46 are arranged at the other plate 43 similarly to the plates 41, 42. As shown in FIG. 3, the light emitters 46 are arranged at equal intervals in the longitudinal direction of the plates 41, 42, 43. As shown in FIG. 4, the light emitters 46 are arranged at substantially the center of each of the plates 41, 42, 43 in a width direction.

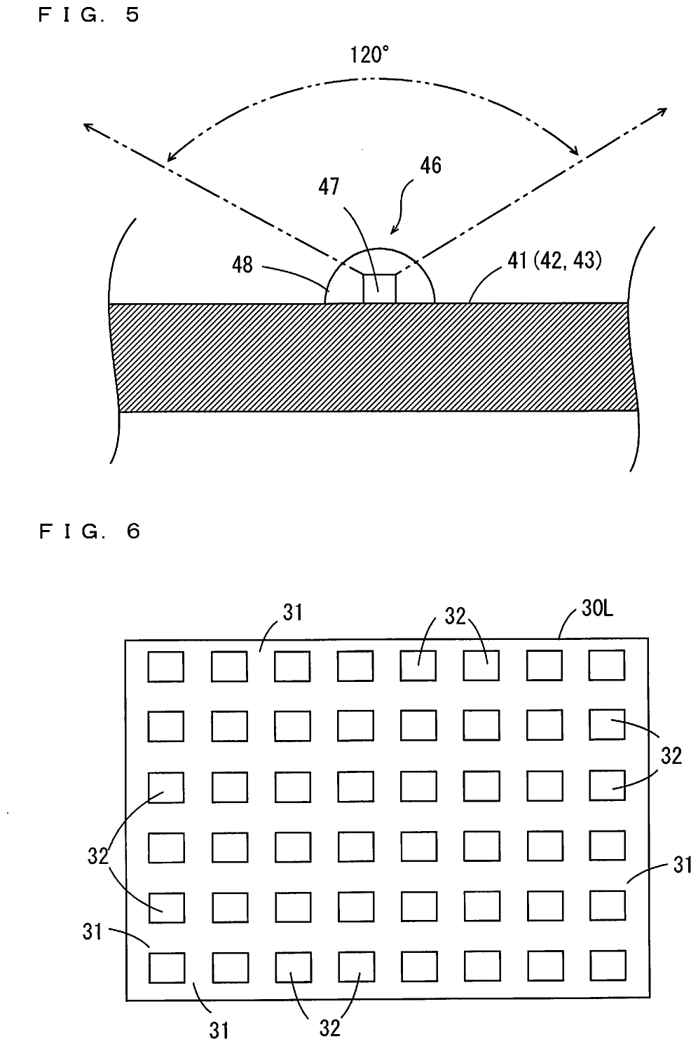

FIG. 5 is an enlarged view of the light emitter 46 shown in FIG. 4. As shown in FIG. 5, the light emitter 46 includes an LED element 47 and a lens 48 that covers the LED element 47. The LED element 47 of the present embodiment can emit ultraviolet rays. Specifically, in the present embodiment, the LED element 47 that emits ultraviolet rays having a wavelength of 285 nm is used. The lens 48 is used to diffuse the ultraviolet rays emitted from the LED element 47. In the present embodiment, the lens 48 can diffuse the ultraviolet rays at a diffusion angle of 120° with respect to a light axis of the LED element 47 as shown in FIG. 5.

Each of the connection portions 45 provided at both ends of the LED supporter 40 has an attachment member for attaching the germicidal lamp 4 to each of the upper wall 30U, the lower wall 30D, the right wall 30R, the left wall 30L, the door (front wall) 30F and the rear wall 30B. Each of the six germicidal lamps 4 is attached to the upper wall 30U, the lower wall 30D, the right wall 30R, the left wall 30L, the door (front wall) 30F and the rear wall 30B with the attachment members.

Each of the connection portions 45 provided at both ends of the LED supporter 40 further has a connector that electrically connects the germicidal lamp 4 to the controller 215. A power cable (not shown) is connected to the connector. The inner casing 3 is stored in the outer casing 2, and then the power cable is connected to the controller 215 via an opening (not shown) provided in the inner casing 3.

### (4) Configuration of Inner Wall of Inner Case

FIG. 6 is a diagram showing the configuration of the inner wall of the left wall 30L. The configuration of the inner wall of each of the upper wall 30, the lower wall 30D, the right wall 30R, the door (front wall) 30F and the rear wall 30B is similar to that of the left wall 30L. Therefore, the configuration of the inner wall of the inner casing 3 is described with the left wall 30L used as an example.

As shown in FIG. 6, the inner wall of the left wall 30L includes a first region 31 formed to have a grid pattern. The first region 31 is a region to which ink mixed with titanium oxide (TiO₂) is applied. That is, titanium oxide is used as a photocatalyst in the first region 31. In this manner, the inner wall of the left wall 30L is treated with a material including titanium oxide. Further, the inner wall of the left wall 30L includes a plurality of oblong second regions 32 that are arranged perpendicularly and horizontally. The second region 32 is a region to which a gold thin film is attached. That is, in the inner wall of the left wall 30L, regions to which the ink mixed with titanium oxide is applied are arranged at positions above, below and at the right and left of the oblong regions to which a gold foil is attached.

Similarly, each of the inner walls of the upper wall 30U, the lower wall 30D, the right wall 30R, the door (front wall) 30F and the rear wall 30B is constituted by the first region 31 and the second region 32 as shown in FIG. 6. In this manner, the inner walls of the upper wall 30U, the lower wall 30D, the right wall 30R, the left wall 30L, the door (front wall) 30F and the rear wall 30B are treated with a material including titanium oxide. Thus, the inner wall of each wall is irradiated with the ultraviolet rays emitted from the above-mentioned germicidal lamps 4, whereby an oxidation-reduction reaction in which titanium oxide is used as a catalyst is promoted, and bacteria, virus and so on are decomposed. Because the second regions 32 to which a gold foil is attached are arranged in the vicinity of the first region 31 containing titanium oxide, the oxidation-reduction reaction in which titanium oxide is used as a catalyst is more sufficiently promoted.

Further, the ink containing titanium oxide is applied to the inner walls of the upper wall 30U, the lower wall 30D, the right wall 30R, the left wall 30L, the door (front wall) 30F and the rear wall 30B, whereby the ultraviolet rays emitted from the above-mentioned germicidal lamps 4 can be efficiently reflected from the inner wall of each wall. Further, because the second regions 32 to which a gold foil is attached are arranged in the vicinity of the first region 31 containing titanium oxide, reflection of ultraviolet rays from the inner wall is further promoted.

### (5) Method of Using Sterilizer

The method of using the sterilizer 1 that is configured as described above will be described. First, the user opens the door 20F and the door 30F and stores an object to be sterilized in the sterilization chamber 5. After storing the object to be sterilized in the sterilization chamber 5, the user closes the door 20F and the door 30F, operates the ON-OFF button 211 in a rotational manner and designates the sterilization period of time. Then, the user depresses the ON-OFF button 211 to provide an instruction for starting the sterilization process. The controller 215 has the function for detecting the door 20F and the door 30F being closed. In a case where the door 20F and the door 30F are closed, the sterilization process starts by the instruction provided by an operation of the ON-OFF button 211.

When the controller 215 starts the sterilization process, electric power is supplied to the six germicidal lamps 4 from the battery 216. Thus, the germicidal lamps 4 emit ultraviolet rays.

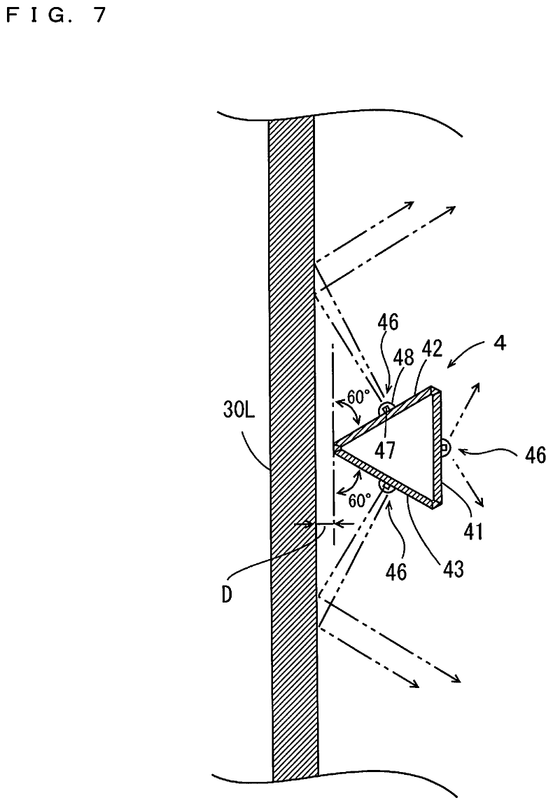

FIG. 7 is a diagram showing the emission directions of the ultraviolet rays emitted from the germicidal lamps 4 attached to the left wall 30L of the inner casing 3. In FIG. 7, the two-dots and dash arrows indicate the emission directions of the ultraviolet rays emitted from the light emitters 46. As described with reference to FIG. 6, the inner wall of the left wall 30L includes the first region 31 treated with titanium oxide and the second regions 32 in which a gold foil is arranged.

As shown in FIG. 7, a plate 41 of the germicidal lamp 4 is arranged substantially in parallel with the inner wall of the left wall 30L. The angles formed by the respective plate surfaces (the attachment surfaces of the LED elements 47) of the plates 42, 43 of the germicidal lamp 4 and the inner wall of the left wall 30L are substantially and respectively 60°. That is, the angles formed by the respective plate surfaces of the plates 42, 43 and the inner wall of the left wall 30L are smaller than 90°. Thus, the ultraviolet rays emitted from the light emitters 46 attached to the plates 42, 43 are emitted from the light emitters 46 and then hit the inner wall of the left wall 30L. In the present embodiment, the distance D between the germicidal lamp 4 and the left wall 30L is adjusted to substantially 1.0 cm as shown in FIG. 7. Therefore, the ultraviolet rays emitted from the light emitters 46 attached to the plates 42, 43 hit the left wall 30L immediately after being emitted from the light emitters 46.

As described above, the first region 31 and the second regions 32 are provided in the inner wall of the left wall 30L. The inner wall of the left wall 30L is irradiated with the ultraviolet rays emitted from the light emitters 46, whereby an oxidation-reduction reaction in which titanium oxide is used as a catalyst is promoted. Thus, an object to be sterilized that is in the vicinity of the left wall 30L is sterilized.

Further, the angles formed by the respective plate surfaces of the plates 42, 43 and the left wall 30L are substantially and respectively 60°, whereby the ultraviolet rays emitted from the light emitters 46 attached to the plates 41, 42 hit the left wall 30L to be reflected. In particular, because the distance between the germicidal lamp 4 and the left wall 30L is substantially 1.0 cm and small in the present embodiment, the ultraviolet rays emitted from the light emitters 46 are mostly reflected from the left wall 30L. Further, because the first region 31 and the second regions 32 are provided in the inner wall of the left wall 30L, reflection of the ultraviolet rays is promoted.

Generally, the traveling distance of ultraviolet rays from the light emitting point is not long. However, in the present embodiment, the ultraviolet rays emitted from the light emitters 46 are reflected from the inner wall of the left wall 30L as described above. Thus, the travelling distance is largely increased. The inventors of the present invention discovered through an experiment that the traveling distance of ultraviolet rays was largely increased by reflection. Thus, the ultraviolet rays reflected from the inner wall of the left wall 30L are emitted toward the center or the other walls of the sterilization chamber 5.

While the germicidal lamps 4 attached to the left wall 30L of the inner casing 3 are described in FIG. 7, the emission state of the ultraviolet rays emitted from the germicidal laps 4 attached to the other walls such as the upper wall 30U, the lower wall 30D, the right wall 30R, the door (front wall) 30F and the rear wall 30B is also similar. The germicidal lamps 4 are also attached to the upper wall 30U, the lower wall 30D, the right wall 30R, the door (front wall) 30F and the rear wall 30B similarly to the way the germicidal lamps 4 are provided on the left wall 30L shown in FIG. 7. Also in the upper wall 30U, the lower wall 30D, the right wall 30R, the door (front wall) 30F and the rear wall 30B, the oxidation-reduction reaction in which titanium oxide is used as a catalyst is promoted in the inner walls similarly to the case of the left wall 30L. The oxidation-reduction reaction is promoted in the inner walls of the six walls, and the object to be sterilized is sterilized.

Further, the ultraviolet rays reflected from the inner walls of the six walls travel in the sterilization chamber 5 and further repeat being reflected from the inner walls of the other walls. Thus, the object to be sterilized is sterilized while the ultraviolet rays are repeatedly reflected in the sterilization chamber 5.

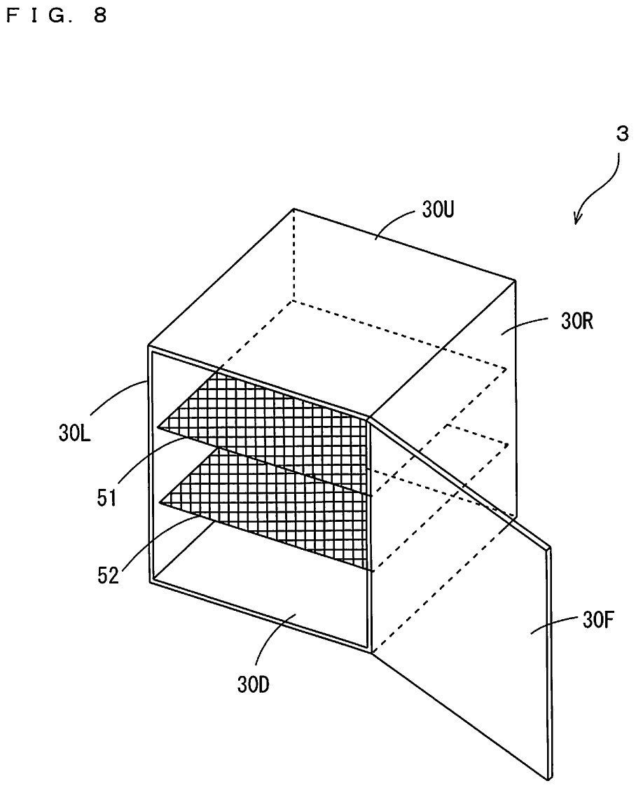

FIG. 8 is a diagram showing the inside of the inner casing 3 to which two netted racks 51, 52 on which objects to be sterilized are to be placed are attached. It is possible to arrange the objects to be sterilized in a multiple levels in the sterilization chamber 5 by placing the objects to be sterilized on the netted racks 51, 52. In FIG. 8, the germicidal lamps 4 in the inner casing 3 are not shown.

As described above, the sterilizer 1 of the present embodiment includes the inner casing 3 in which the inner walls are treated with a material including titanium oxide and the LED elements 47 for emitting ultraviolet rays attached to the inner walls of the inner casing 3. Thus, the object to be sterilized in the sterilization chamber 5 can be sterilized by the ultraviolet rays reflected from the inner walls of the inner casing 3.

Because the sterilizer 1 of the present embodiment uses the LED elements 47, the electric power required for the sterilization process can be significantly reduced as compared to a conventional sterilizer that requires a high pressure and a high temperature as conditions. For example, a product that requires power consumption of 0.7 W can be used as LED elements 47. In the case of the present embodiment, the number of LED elements 47 attached to one plate is four, and the three plates 41, 42, 43 are used. Therefore, the power consumption is 0.7 W×4×3=8.4 W. For example, in comparison with an autoclave that requires power consumption of about 1 kW to 2 kW, the power consumption is drastically reduced.

As described above, the sterilizer 1 of the present embodiment is extremely light since being constituted by the LED elements 47 and the plates 41, 42, 43 supporting the LED elements 47, the controller 215 and the battery 216. As described above, since the power consumption of the sterilizer 1 is extremely low, the capacity of the battery 216 may be small. Therefore, a light battery can be used as the battery 216. Further, because a heavy component such as a motor is not required, the entire device can be light. Thus, the device is favorable to carry for outdoor use. The device is extremely useful in a case where being used in outdoor hospitals provided in regions where outbreak of bacterial infection occurs, for example.

Further, since the power consumption of the sterilizer 1 of the present invention is low, the sterilizer 1 can meet the requirement for being used with the power generated using solar panels. Thus, the device is extremely effective for outdoor use.

The sterilizer 1 of the present invention can kill bacteria and virus using ultraviolet rays. Ultraviolet rays are considered to destroy protoplasm of bacteria, and bacteria is killed by ultraviolet rays regardless of its type. Further, ultraviolet rays kill virus by destroying an envelope and capsids of virus. Although the type of bacteria that is resistant to the high-temperature and the high-pressure may be generated in the future, bacteria can be effectively killed with use of ultraviolet rays.

The sterilizer 1 of the present embodiment, described above, can be used for sterilization of surgical tools at hospitals, sterilization of equipment such as towels in hospitals, sterilization of tableware in households, sterilization of clothing, shoes and so on in households or laboratory instruments in universities and research institutions, for example. Further, with its low power consumption, the device is suitable for use in mobile hospitals such as boats and outdoor hospitals in Africa, etc. Further, because being able to destroy molds, the sterilizer 1 of the present embodiment can be used as an air cleaner in households, facilities, etc.

### (6) Other Embodiments

In the above-mentioned embodiment, the inner walls of the upper wall 30U, the lower wall 30D, the right wall 30R, the left wall 30L, the door (front wall) 30F and the rear wall 30B include the second regions 32. Further, gold thin films were used for the second regions 32. Another metal that can promote an oxidation-reduction reaction caused by titanium oxide can be used for the second regions 32. For example, platinum can be used instead of gold.

In the above-mentioned embodiment, as shown in FIG. 4, the LED supporter 40 is constituted by the three plates 41, 42, 43, and the three plates 41, 42, 43 are assembled such that the cross section that is cut perpendicularly to the longitudinal direction of the LED supporter 40 is an equilateral triangle. As another embodiment, three plates 41, 42, 43 may be constituted such that the cross section of a LED supporter 40 is a triangle other than an equilateral triangle. For example, a LED supporter 40 may be constituted such that the plate 41 arranged in parallel with the wall of the inner casing 3 is wider than the other plates 42, 43. Further, the respective angles formed by the respective plates 42, 43 and the inner wall of the inner casing 3 may be smaller than 60°. Alternatively, a LED supporter 40 may be constituted by four or more plates, and its cross section may form a polygon such as a square or a pentagon. Even in this case, the LED supporter 40 is constituted such that the angle formed by some respective plates out of the plurality of plates and the inner wall of the inner casing 3 is smaller than 90°. Thus, the inner wall of the inner casing 3 can be effectively irradiated with ultraviolet rays emitted from the light emitters 46.

While one germicidal lamp 4 is attached to one inner wall of the inner casing 3 in the above-mentioned embodiment, this is merely one example. A plurality of germicidal lamps 4 may be attached to one inner wall.

In the above-mentioned embodiment, the distance D between the germicidal lamp 4 and the left wall 30L is adjusted to substantially 1.0 cm. The distance D between the germicidal lamp 4 and the left wall 30L is not limited to this, and the distance desirably allows effective reflection of ultraviolet rays. For example, the distance D between the germicidal lamp 4 and the left wall 30L is about 0 to 5.0 cm.

While the LED element 47 that emits ultraviolet rays having a wavelength of 285 nm is used in the above-mentioned embodiment, an LED element that emits ultraviolet rays having a shorter wavelength may be used. For example, it is possible to enhance sterilization effects by using an LED element that emits ultraviolet rays having a wavelength of 250 nm.

While the embodiments of the present invention are described above, the above-mentioned embodiments are merely examples of embodiments of the present invention. While preferred embodiments of the present invention have been described above, it is to be understood that variations and modifications will be apparent to those skilled in the art without departing the scope and spirit of the present invention. The scope of the present invention, therefore, is to be determined solely by the following claims.

I/We claim:

1. A sterilizer comprising:
a casing having an inner wall that is treated with a material including titanium oxide; and
an LED element for emission of ultraviolet rays that is attached to the inner wall of the casing.

2. The sterilizer according to claim 1, wherein
a metal thin film is further formed on part of the inner wall.

3. The sterilizer according to claim 2, wherein
the metal includes gold.

4. The sterilizer according to claim 1, wherein
the sterilizer further includes an LED supporter to which the LED element is attached, and
an angle formed by an attachment surface of the LED supporter to which the LED element is attached and the inner wall is smaller than 90°.

5. The sterilizer according to claim 4, wherein
the LED supporter include a plurality of plates, and
a cross section of the LED supporter is formed to be a polygon by the plurality of plates.
